(19) United States
(12) Patent Application Publication
(10) Pub. No.: US 2025/0080639 A1
(43) Pub. Date: Mar. 6, 2025

(54) METHOD FOR INCREASING THE USER COMFORT OF A VEHICLE OCCUPANT AND VEHICLE

(71) Applicant: MERCEDES-BENZ GROUP AG, Stuttgart (DE)

(72) Inventors: Gudrun SCHÖNHERR, Holzgerlingen (DE); Svetlana MARIAN, Böblingen (DE)

(21) Appl. No.: 18/725,855

(22) PCT Filed: Dec. 1, 2022
(86) PCT No.: PCT/EP2022/084003

§ 371 Date: Jul. 1, 2024

(30) Foreign Application Priority Data
Jan. 3, 2022 (DE) 10 2022 000 029.6

(51) Int. Cl. H04M 1/72409; H04M 1/72484; H04M 19/04

(52) U.S. Cl. CPC H04M 1/724098; H04M 1/72484; H04M 19/047

(57) ABSTRACT

User comfort of a vehicle occupant staying in a vehicle is increased by conveying haptic feedback to the vehicle occupant using an actuator arranged in the vehicle. The actuator is controlled by a computer unit of the vehicle depending on information received by a mobile terminal. A program is run on a first mobile terminal, which emits control signals to actuate a haptic feedback actuator of the first mobile terminal. The first mobile terminal communicates directly or indirectly via a second mobile terminal with the computer unit of the vehicle. Control signals emitted by the program run on the first mobile terminal are conveyed to the computer unit. The computer unit of the vehicle controls the at least one actuator arranged in the vehicle for generating the haptic feedback depending on the control signals received by the first mobile terminal.

### BACKGROUND AND SUMMARY OF THE INVENTION

Exemplary embodiments of the invention relate to a method for increasing the user comfort of a vehicle occupant while staying in a vehicle and to a vehicle for carrying out the method.

During the journey with a vehicle, situations can emerge for vehicle occupants which lead to stress, such as waiting in a traffic jam, for example. Various vehicle functions, such as listening to the radio, streaming music, playing on a video game console, or watching videos via an infotainment system of the vehicle can be used to distract and to reduce stress. Some vehicles also offer the function of carrying out relaxation exercises, for example breathing exercises. Here, the vehicle occupants are guided by the vehicle to carry out the relaxation exercise. For this, the vehicle can emit visual information via a display and/or emit acoustic information via loudspeakers. Carrying out the relaxation exercise can be supported by playing relaxing music. However, to carry out such a relaxation exercise, the vehicle occupant has to firstly manually start the corresponding relaxation program, which is associated with effort for the vehicle occupant. This often leads to such relaxation functions only being used rarely.

Furthermore, so-called wearables, such as smart wrist straps or smart watches, are known, which monitor the vital state of the wearer and emit recommendations for carrying out relaxation exercises as needed. Here, the wearer is guided by the wearable to accurately carry out the relaxation exercise. Thus, the wearable emits a piece of haptic feedback, for example by means of an actuator, which, when carrying out a breathing exercise, is intended to prompt breathing in and out specifically. The piece of haptic feedback is here adjusted to the breath frequency, such that the wearer is to breathe in and out exactly when a certain piece of haptic feedback is emitted via the wearable or fails to appear. However, the disadvantage here is that a piece of haptic feedback emitted via a wearable can often not be sufficiently perceived by a wearer, for example because the wearer is wearing the wearable too loosely. In particular when operating a vehicle, the wearable can slip out of place on the wrist as a result of using the steering wheel, which makes the use of wearables for emitting haptic feedback in the vehicle unpractical.

Training programs for carrying out relaxation exercises can also be downloaded in the form of so-called apps on a smartphone or tablet computer. Here, the corresponding mobile terminal can also be coupled to an appropriate wearable, such that the wearable receives control commands for emitting the haptic feedback from the mobile terminal.

Furthermore, vehicle seats with integrated actuators are known which can be used for massaging vehicle occupants, for example.

In addition, US 2016/0107570 A1 discloses a system and a method for improving the continuous attention in vehicles by emitting haptic feedback. The system comprises at least one sensor, which monitors the surroundings and/or a state of a vehicle and/or a vehicle occupant. In doing so, adjacent traffic participants, for example, and their relative position to the vehicle can be recognized, and/or a health and/or attention state of vehicle occupants can be recorded. The vehicle can comprise one or more actuators, which emit haptic feedback. These can be integrated in the vehicle seat, in the steering wheel or in a seatbelt, for example. Wearables can also be used for emitting haptic feedback. Depending on the parameters recorded by the sensor, the system forwards haptic feedback onto a vehicle occupant. Thus, it can be communicated to the driver, for example, via haptic feedback that a further vehicle is in a blind spot or that the driver is currently crossing over a lane marking. The haptic feedback can also comprise direction information and have a movement directed in a certain orientation, for example. The driver can also be “nudged” via the haptic feedback, which improves their attention.

Exemplary embodiments of the present invention are directed to a method for increasing the user comfort of a vehicle occupant while staying in a vehicle, by means of which the user comfort can be increased particularly reliably and comfortably.

In a method for increasing the user comfort of a vehicle occupant when staying in a vehicle of the kind specified at the start, a piece of haptic feedback is conveyed to the vehicle occupant by means of at least one actuator arranged in the vehicle, wherein the actuator is controlled by a computer unit of the vehicle depending on information received by a mobile terminal, wherein according to the invention:
a program is run on a first mobile terminal, which emits control signals for actuating a haptic feedback actuator of the first mobile terminal;
the first mobile terminal communicates directly with the computer unit of the vehicle or indirectly via a second mobile terminal with the computer unit of the vehicle, wherein the control signals emitted by the program run on the first mobile terminal are conveyed to the computer unit; and
the computer unit of the vehicle controls the at least one actuator arranged in the vehicle for generating the haptic feedback depending on the control signals received by the first mobile terminal.

The first mobile terminal can be a wearable, such as a smart bracelet or a smartwatch, or also a tablet computer or a smartphone. Programs, also referred to as apps, can be run on such a mobile terminal, which cause a piece of feedback to be emitted via the respective mobile terminal. These control signals can be tapped by the computer unit of the vehicle and used to control an actuator installed in the vehicle for generating haptic feedback. In other words, the first mobile terminal remote controls the haptic feedback emitted via the vehicle. This has crucial advantages for a vehicle occupant. Thus, a piece of haptic feedback emitted via actuators arranged in the vehicle can be perceived more clearly by the vehicle occupant than a piece of haptic feedback emitted via a wearable or smartphone. In addition, emitting the haptic feedback is carried out automatically, prompted by the program run on the first mobile terminal. In doing so, vehicle occupants can be comfortably reminded of certain things or prompted to carry out certain activities.

If the first mobile terminal is a wearable, then the program, i.e., the app, can also be run on a second mobile terminal. In this case, the second mobile terminal is a tablet computer or a smartphone. The first mobile terminal and the second mobile terminal are then coupled to each other, which enables a communication of a wearable with the computer unit of the vehicle, since the computer unit of the vehicle is also coupled to the tablet computer or smartphone.

To couple the mobile terminals to one another and to the computer unit of the vehicle, any digital communication techniques can be used, such as cable-bound or wireless communication technologies. For example, a tablet computer or smartphone can be plugged into the computer unit with a USB cable. For a wearable to communicate with a smartphone or tablet computer and for a tablet computer or smartphone to communicate with the computer unit, Bluetooth, Wi-Fi, NFC ultra-broadband communication, for example, or similar can be used.

Emitting the haptic feedback in the vehicle can take place both during the journey with the vehicle and when stopped. The corresponding actuators can be arranged in the vehicle at any point, which enables the haptic feedback to be conveyed to the vehicle occupants so long as a corresponding vehicle occupant touches a corresponding point at which a corresponding actuator is arranged at given time windows. For example, one or more actuators can be integrated into a vehicle seat, into the steering wheel of the vehicle, into operating elements, into a seatbelt or similar. As actuators, any established actuators can be used for generating a piece of haptic feedback, such as electric drives with eccentric mass, piezo elements, and similar. The haptic feedback has a modulation. This means that it is variable in its strength, frequency, duration, and similar. The haptic feedback emitted in the vehicle can correspond to the modulation of the haptic feedback provided originally for emitting via the haptic feedback actuator of the first mobile terminal.

The haptic feedback can be emitted to a person driving the vehicle, a passenger and/or even to passengers in the backseats. Here, the same piece of haptic feedback or an individual piece of haptic feedback can be impressed on the vehicle occupants.

An advantageous development of the method provides that the program run on the first mobile terminal initiates control signals to be emitted depending on a monitoring of incoming calls, a time monitoring, and/or an instruction for carrying out breathing exercises. If the vehicle occupant is carrying a smartphone on them that receives a phone call, then the smartphone typically vibrates and/or transmits a caller ID to a wearable, such as a smartwatch. The smartwatch can then also vibrate. The actuators installed in the vehicle are then also actuated via the coupling of the smartphone to the computer unit of the vehicle. If the vehicle occupant is not carrying the smartphone on their person, but instead has placed it down in a charging cradle, for example, and if the corresponding vehicle occupant is not wearing a wearable or does not feel the haptic feedback emitted by the wearable, then the vehicle occupant can be made aware of the phone call via the haptic feedback generated by the vehicle.

The smartphone or the wearable can also emit haptic feedback depending on the time of day, a countdown timer, or a stopwatch. Thus, for example after a set period of time, a smartwatch can emit haptic feedback every 15 minutes, for example in the manner of the chime of a church clock. For example, 15 minutes after a full hour, haptic feedback can be emitted as a single beat, 30 minutes after a full hour as two beats, and 45 minutes after a full hour as three beats. This enables the vehicle occupants to track the elapsed time without the corresponding vehicle occupant having to direct their gaze to a display.

In a quite particularly preferred design of the method according to the invention, the program is a program for carrying out relaxation exercises, such as breathing exercises. Such a program guides a person to focus on their breathing and thus to deliberately breathe in and out, or to hold their breath. For example, a breathing pattern can provide that the person is to breathe in for four seconds, hold their breath for seven seconds and breathe out for eight seconds or, as with so-called “box breathing”, is to breathe in and out for four seconds in each case, with respective breathing pauses of four seconds in between.

These breathing techniques are typically augmented by visual information on a display of the smartphone, tablet computer, and/or wearable. Analogously, a piece of haptic feedback appropriate for breathing in, holding one's breath and/or breathing out is emitted via the wearable. For example, a piece of surging haptic feedback can be conveyed to symbolize a breathing-in process, a piece of absent haptic feedback to symbolize holding one's breath, and a piece of subsiding haptic feedback to symbolize a breathing-out process. As a result of emitting the haptic feedback via the vehicle, it can be perceived quite particularly clearly and comfortably by the vehicle occupants.

By means of the method according to the invention, several vehicle occupants are additionally able to simultaneously carry out breathing training. Here, it is sufficient when a single vehicle occupant carries a corresponding first mobile terminal on them. The vehicle, that is to say the computer unit, can then control several actuators in the vehicle at the same time, such that several people can participate in the breathing exercise at the same time. In general, it is also conceivable that the breathing exercise is adjusted individually to the different vehicle occupants, such that each vehicle occupant does individual training.

The haptic feedback can also be emitted in the vehicle in order to “nudge” a vehicle occupant, whereby the vehicle occupant is prompted to carry out a breathing exercise. Subsequently, the guidance to carry out the breathing exercise can be carried out visually and/or acoustically, and the actuator(s) can be used in the vehicle to emit the haptic feedback in the form of a massage program during the breathing exercise.

Corresponding to a further advantageous design of the method, a different piece of haptic feedback is emitted in the vehicle for different contacts. This makes it possible for the vehicle occupant to identify different callers by sensing an individual piece of haptic feedback. For example, the haptic feedback for a first contact can correspond in terms of its amplitude to a sine wave and for a second contact to a sawtooth impulse with a pause of one second after every three sawteeth.

Corresponding to a further advantageous design of the method according to the invention, the haptic feedback emitted in the vehicle is adjusted in terms of its modulation compared to the haptic feedback provided for emitting via the first mobile terminal. As already mentioned, the haptic feedback emitted in the vehicle can correspond to the haptic feedback emitted via the first mobile terminal. However, it can also differ here. The possibilities for emitting the haptic feedback via the first mobile terminal are limited because a mobile terminal typically only has one actuator for generating haptic feedback. However, several actuators can be arranged in the vehicle, in particular in different positions, whereby a particularly comprehensive and exciting haptic feedback experience can be generated in the vehicle. While a vibration only on the wrist of the vehicle occupant can be emitted, for example, via a smartwatch when carrying out a breathing exercise, a direction can also be impressed upon a piece of haptic feedback, for example via actuators integrated in a vehicle seat. For example, the haptic feedback can move upwards in the backrest of a vehicle seat, in order to symbolize breathing in and can move downwards in order to symbolize breathing out. In general, the haptic feedback emitted in the vehicle can be implemented differently, for example in terms of its frequency, duration, strength, direction, and similar, to the haptic feedback originally emitted via the first mobile terminal.

Corresponding to a further advantageous design of the method, in addition to the haptic feedback emitted in the vehicle, information depending on the context of the control signals received by the first mobile terminal is visually depicted on a display in the vehicle. If the vehicle occupant receives a phone call, for example, then contact information and an icon to accept the call or reject the call can be depicted on a display in the vehicle. Analogously, the time, a stopwatch, or a countdown timer can be depicted on the display. The guidance or information depicted on the display of the smartphone or tablet computer for carrying out a breathing exercise can also be transferred to the display of the vehicle. Such guidance often comprises an animation, such as a surging or subsiding geometric object.

A further advantageous design of the method further provides that the vehicle occupant sets the manner of how the haptic feedback is emitted in the vehicle and/or how the information is visually depicted in the vehicle. In doing so, the vehicle occupant can satisfy their personal preferences. For example, the vehicle occupant can set that the haptic feedback emitted in the vehicle is to be distinguished from the haptic feedback emitted via the wearable or the smartphone. Here, they can also establish how a different modulation of the haptic feedback emitted in the vehicle is to be seen exactly. The vehicle occupant can also adjust the depiction of the information on the display in the vehicle to their preferences. For example, they can select whether a piece of information is to be depicted as text and/or graphically and, for example, establish a position of a corresponding graphic on the display.

Preferably, when the computer unit of the vehicle receives control signals from the first mobile terminal, the computer unit specifies a request for inputting an affirmative operating action to the vehicle occupant and only emits the haptic feedback in the vehicle when the vehicle occupant inputs the affirmative operating action. Thus, smartwatches or smart wristbands often have a reminder function that a certain activity is to be carried out after a set time, for example once per hour. Such reminders provide, for example, standing up and walking around, turning away from a screen activity, carrying out a breathing exercise or similar. Here, the reminder can be supported by emitting haptic feedback. During the journey, a person driving the vehicle is often focused on driving occurrences. There is therefore the possibility that the person driving the vehicle is stressed and forgets to assume a relaxed posture. For example, such a reminder can have a request for carrying out a relaxation technique, for example a breathing exercise. Thus, after receiving such a reminder, the computer unit of the vehicle can ask the vehicle occupant whether they would like to carry out a corresponding breathing exercise. Thus, the frequency with which unused vehicle functions are used can be increased. Here, the breathing exercise can correspond to the breathing exercise that can be run on the first mobile terminal, or the person driving the vehicle or the vehicle occupant can select one of several breathing exercises provided by the computer unit of the vehicle. In general, it is naturally also possible here that the breathing exercise is started automatically directly upon receiving the reminder to carry out the relaxation technique. Otherwise, a touch display can be used in the vehicle to input a corresponding operating action or carry out an input by means of speech commands.

In a vehicle with a computer unit and a communication interface, the computer unit and the communication interface are set up, according to the invention, to carry out a method described above. The vehicle can be any vehicle, such as a passenger car, lorry, transporter, bus, or similar. The computer unit can be a central on-board computer, a telematics unit, or a different control device of a vehicle subsystem. The computer unit can create a communication connection to the first mobile terminal and/or the second mobile terminal with the aid of the communication interface. The computer unit can also create a communication connection to a central computer unit, that is to say a cloud server, via the communication interface, for example by mobile telephony. New breathing exercises and/or different haptic feedback, for example, differing in terms of its modulation can be retrieved via the Cloud server.

Further advantageous designs of the method according to the invention for increasing the user comfort of a vehicle occupant also emerge from the exemplary embodiments, which are described below in more detail with reference to the figures.

### BRIEF DESCRIPTION OF THE DRAWING FIGURES

Here are shown:

FIG. 1 a schematic view of the components required to carry out a method according to the invention for increasing the user comfort of a vehicle occupant; and

FIG. 2 a schematized flowchart of a method according to the invention.

### DETAILED DESCRIPTION

When travelling with a vehicle, stressful situations can occur for vehicle occupants. To reduce a stress level of a respective vehicle occupant, relaxation techniques such as breathing exercises can be carried out. Using a method according to the invention, carrying out relaxation exercises in the vehicle can be supported by emitting haptic feedback in the vehicle, whereby the user comfort for the vehicle occupant when inside the vehicle can be improved. Moreover, the haptic feedback emitted in the vehicle can be used to identify incoming callers and illustrating elapsed time.

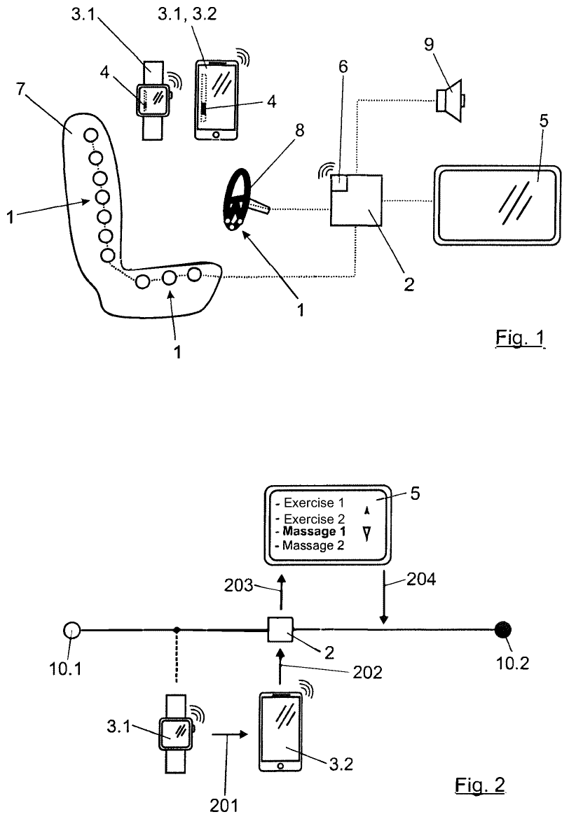

The components used to carry out a method according to the invention are depicted in FIG. 1. The haptic feedback is emitted via one or more actuators 1 arranged in the vehicle. For example, one or more actuators 1 can be integrated into a vehicle seat 7 and/or the steering wheel 8 of the vehicle. The actuator(s) 1 are controlled by a computer unit 2, which for its part receives control commands from a first mobile terminal 3.1 via a communication interface 6. The first mobile terminal 3.1 can communicate directly with the computer unit 2 or indirectly via a second mobile terminal 3.2.

A program is run on the first mobile terminal 3.1, for example caller recognition, time monitoring, and/or a program for guiding a relaxation technique, in particular a breathing exercise. The program emits control commands for emitting a piece of haptic feedback via a haptic feedback actuator 4 integrated into the first mobile terminal 3.1. The corresponding control signals are tapped by the computer unit 2 and are used to imprint the haptic feedback onto the actuators 1.

Emitting haptic feedback in the vehicle can be augmented by visual information via a display 5 and/or acoustic information via a loudspeaker 9 in the vehicle. For example, an acoustic instruction for carrying out a breathing exercise can be emitted via the loudspeaker 9 and/or a breathing exercise can be made even more pleasant by emitting relaxing sounds.

FIG. 2 shows a schematized flowchart of a method according to the invention. During the journey of the vehicle from a starting point 10.1 to a target point 10.2, the first mobile terminal 3.1, here in the form of a smartwatch, registers that carrying out a relaxation exercise is required for a vehicle occupant. For example, the smartwatch can establish this by it establishing a set period of time having elapsed, for example an hour, or ascertaining an increased stress level of the vehicle occupant by evaluating vital signs. The smartwatch then forwards the recommendation to carry out the relaxation technique to a second mobile terminal 3.2, here in the form of a smartphone, for example, for example via Bluetooth, as indicated by arrow 201.

In turn, the smartphone forwards this information to the computer unit 2 of the vehicle, as indicated by arrow 202.

Subsequently, indicated by arrow 203, the computer unit 2 initiates emitting a request to the vehicle occupant via the display 5 of the vehicle that carrying out a relaxation technique is recommended. The vehicle occupant can choose via the display 5, for example designed as a touchscreen, how to further progress now. Thus, the vehicle occupant can select via the display 5 whether the breathing exercise is to be carried out according to the suggestion of the smartwatch, wherein the actuators 1 of the vehicle are to be controlled analogously to the haptic feedback emitted via the smartwatch, or whether the actuators 1 are to be controlled in the vehicle according to a deviating modulation. The vehicle occupant can also select that the breathing exercise itself is instructed exclusively by the smartwatch, and the breathing exercise in the vehicle itself is augmented by a massage. Here, the vehicle occupant can select on the display 5 one of various massage or workout programs, in which in each case the actuators 1 are controlled differently in the vehicle. The vehicle occupant can also select whether the depiction of instructing or helpful information is to be carried out on the display 5 and/or emitting acoustic instructions and/or relaxing sounds are to be carried out via the loudspeaker 9. Here, the vehicle occupant can also establish that, when receiving a reminder again to carry out a relaxation technique by the smartwatch, the corresponding selection made by them is selected automatically by the vehicle the next time. Now, the corresponding breathing exercise is carried out and ends, indicated by arrow 204.

Thanks to the reminder emitted by the smartwatch or thanks to monitoring the stress level of the vehicle occupant, the vehicle occupant is required at the correct moment to use a relaxation technique. By emitting the haptic feedback in the vehicle and optionally augmented by the information emitted on the display 5 and/or the loudspeaker 9, the corresponding relaxation technique can be carried out quite particularly comfortably in the vehicle. This considerably increases the user comfort of the vehicle occupant.

Although the invention has been illustrated and described in detail by way of preferred embodiments, the invention is not limited by the examples disclosed, and other variations can be derived from these by the person skilled in the art without leaving the scope of the invention. It is therefore clear that there is a plurality of possible variations. It is also clear that embodiments stated by way of example are only really examples that are not to be seen as limiting the scope, application possibilities or configuration of the invention in any way. In fact, the preceding description and the description of the figures enable the person skilled in the art to implement the exemplary embodiments in concrete manner, wherein, with the knowledge of the disclosed inventive concept, the person skilled in the art is able to undertake various changes, for example, with regard to the functioning or arrangement of individual elements stated in an exemplary embodiment without leaving the scope of the invention, which is defined by the claims and their legal equivalents, such as further explanations in the description.

1-8. (canceled)

9. A method for increasing the user comfort of a vehicle occupant in a vehicle, the method comprising:
executing, on a first mobile terminal, a program emitting control signals to actuate a haptic feedback actuator of the first mobile terminal;
communicating, by the first mobile terminal directly or indirectly via a second mobile terminal, with a computer unit of the vehicle the emitted control signals to the computer unit; and
controlling, by the computer unit of the vehicle, at least one actuator arranged in the vehicle to generate haptic feedback depending on the emitted control signals received by the first mobile terminal; and
generating, by the at least one actuator arranged in the vehicle, the haptic feedback.

10. The method of claim 9, wherein the program executed on the first mobile terminal initiates emitting control signals depending on a monitoring of incoming calls, a time monitoring, or an instruction to carry out relaxation exercises.

11. The method of claim 10, wherein a different piece of haptic feedback is emitted in the vehicle for different contacts of incoming calls.

12. The method of claim 9, wherein a modulation of the haptic feedback generated by the at least one actuator in the vehicle is adjusted compared to haptic feedback output by the first mobile terminal based on the emitted control signals.

13. The method of claim 9, wherein, in addition to the haptic feedback generated by the at least one actuator in the vehicle, information depending on a context of the emitted control signals is depicted visually in the vehicle on a display of the vehicle.

14. The method of claim 13, wherein the vehicle occupant sets a manner of how the haptic feedback is generated in the vehicle or how the information is depicted visually in the vehicle.

15. The method of claim 9, wherein when the computer unit of the vehicle receives the emitted control signals from the first mobile terminal, the computer unit outputs a request for inputting an affirmative operating action to the vehicle occupant and emits the haptic feedback in the vehicle only when the vehicle occupant inputs the affirmative operating action.

16. A vehicle comprising:
a computer unit;
at least one actuator coupled to the computer unit; and
a communication interface coupled to the computer unit,
wherein the computer unit is configured to receive, from a first mobile terminal via the communication interface, emitted control signals, wherein the emitted control signals are generated by a program executed on the first mobile terminal to actuate a haptic feedback actuator of the first mobile terminal, and wherein the computer unit receives the emitted control signals either directly from the first mobile terminal or via a second mobile terminal, and
wherein the computer unit is configured to control the at least one actuator to generate haptic feedback depending on the emitted control signals received from the first mobile terminal.
